(19) United States
(12) Patent Application Publication
(10) Pub. No.: US 2012/0046362 A1
(43) Pub. Date: Feb. 23, 2012

(54) ANTIVIRAL AGENT AND CLEANSING AGENT

(75) Inventors: Takayoshi Kawahara, Fukuoka (JP); Maiko Kusaba, Fukuoka (JP); Takemasa Sakaguchi, Hiroshima (JP)

(73) Assignees: HIROSHIMA UNIVERSITY, HIGASHI-HIROSHIMA-SHI (JP); SHABONDAMA SOAP CO., LTD, Kitakyushu-shi (JP)

(21) Appl. No.: 13/264,934

(22) PCT Filed: Apr. 16, 2010
(86) PCT No.: PCT/JP10/56879

§ 371 Date: Oct. 17, 2011

(30) Foreign Application Priority Data
Apr. 17, 2009 (JP) 2009-101458
Nov. 20, 2009 (JP) 2009-265756

(51) Int. Cl. A61K 31/20; A61P 31/14; A61Q 19/10; A61P 31/12

(52) U.S. Cl. 514/558

(57) ABSTRACT

It is an object to provide an antiviral agent that can be used for persons having sensitive skin or on the face, inactivates viruses such as a norovirus and an influenza virus, and is excellent in germicidal properties. Further provided is a cleansing agent that does not lead to environmental pollution since the cleansing agent is easily decomposed in the natural environment, scarcely causes eczema and allergic dermatitis since no germicidal agent is added, and has an antiviral performance. The antiviral agent containing a surface-active agent having a C18 unsaturated alkyl group as an active component. It is not always necessary to lather or rinse off with water like cleansing agents such as medicated soaps since the antiviral agent of the present invention at a very low concentration can inactivate the virus.

### TECHNICAL FIELD

The present invention relates to an antiviral agent that inactivates new influenza viruses (swine-origin influenza virus A (H1N1) that appeared as an epidemic in 2009) in addition to a norovirus, an avian influenza virus and a human influenza virus, and a cleansing agent containing the antiviral agent as an active component.

### BACKGROUND ART

Noroviruses orally infect humans to cause a transmissible gastrointestinal infectious disease (infectious gastroenteritis). A norovirus has no envelope, thus is highly resistant to an invert soap (benzalkonium chloride) and ethanol for disinfection, and is difficult to sterilize. Thus, in order to prevent its infection, it is recommended that persons who cook wash their hands thoroughly to rinse off the virus physically.

Influenza is transmitted by inhaling the influenza virus released by airborne droplets from coughing, sneezing and from saliva of affected patients into an airway such as a nasal cavity and bronchial tube. The released virus adheres to hands, face and clothing, etc. Thus, rubbing eyes by hand and touching the nose are also infection pathways. Thus, it is recommended as prevention against infection to wash hands and face after returning home to physically rinse off the virus adhered to the hands and face, etc.

Those combining polyglycerin fatty acid ester (Patent Literature 1) are disclosed as an aqueous sterilized disinfectant for killing a norovirus, and those combining an organic acid salt and a eucalyptus extract with ethanol (Patent Literature 2) are disclosed as an alcohol-based antiviral agent which inactivates a norovirus. Also, a mixture of an antimicrobial active substance, an anionic surface-active agent having a straight alkyl chain with a chain length of C4 to C12 and a hydrophilic group with a size of 4 angstroms or more, and an anionic surface-active agent having a branched alkyl chain or an unsaturated alkyl chain with a chain length of C4 to C12 (Patent Literature 3) is disclosed as an antimicrobial composition having an antiviral activity.

### CITATION LIST

### Patent Literatures

PTL 1: Japanese Published Unexamined Patent Application No. 2008-156329
PTL 2: Japanese Published Unexamined Patent Application No. 2009-159577
PTL 3: Published Japanese Translation of PCT Application No. 2005-530857

### SUMMARY OF INVENTION

### Technical Problem

However, the above conventional arts have had problems as follows.

(1) The technology disclosed in Patent Literature 1 may inactivate a norovirus, but sometimes causes irritation when rubbed by hands (paragraphs 0052 to 0053 in the publication), and thus has the problem that the technology may not be used for the hands and face of a person having sensitive skin and the surface of the skin may not be cleansed because the technology does not provide a cleansing agent.
(2) The technology disclosed in Patent Literature 2 may inactivate a norovirus, but may not be used for persons who are sensitive to alcohol, and has the problem that the surface of the skin may not be cleansed because the technology does not provide a cleansing agent.
(3) The technology disclosed in Patent Literature 3 may sterilize and inactivate many kinds of bacteria and viruses, but has the problem that the anionic surface-active agent composing the technology is not derived from a natural component, is difficult to decompose in the natural world, and thus leads to environmental pollution.
(4) In many medicated cleansing agents, a germicidal agent is added as an antiviral component, but these additives have sometimes caused eczema and allergic dermatitis.

The present invention has solved the conventional problems described above, and it is an object of the present invention to provide an antiviral agent that inactivates viruses such as a norovirus and an influenza virus and is excellent in germicidal properties without causing irritation even when used for the hands and face of a person having sensitive skin. It is another object of the present invention to provide a cleansing agent having an antiviral performance, which is easily decomposed in the natural environment thus not leading to environmental pollution and is difficult to cause eczema and allergic dermatitis since no germicidal agent is added.

### Solution to Problem

The antiviral agent of the present invention has the following constitutions in order to solve the above conventional problems.

The antiviral agent according to a first aspect of the present invention has the constitution in which a percentage of a summed amount of a laurate soap and a myristate soap is 70% by weight or more based on the total amount of the saturated fatty acid soaps, and the percentage of a stearate soap and the percentage of a palmitate soap are less than 15% by weight based on the total amount of the saturated fatty acid soaps, and the percentage of the oleate soap is 50 to 75% by weight based on the total amount of the soaps.

The following actions are obtained by this constitution.

(1) By carrying out an extensive study and evaluating an inactivation ability of the surface-active agents against viral infectivity, the inventors of the present invention have found a previously unknown attribute that the surface-active agent having the C18 unsaturated alkyl group has high inactivation ability against infectivity of a virus such as an influenza virus and a feline calicivirus, and have found based on this discovery that the surface-active agent having the C18 unsaturated alkyl group is suitable for new use as an antiviral agent.
(2) The surface-active agent having the C18 unsaturated alkyl group reduces the infectivity of an influenza virus and a feline calicivirus to reduce a viral infectivity titer to 1% or less, and has very high inactivation ability against infectivity of an influenza virus and a feline calicivirus.
(3) The surface-active agent having the C18 unsaturated alkyl group may inactivate the virus at a very low concentration. Thus, it is not always necessary to lather and rinse off with water as in the case of the cleansing agent such as medicated soaps.
(4) The soaps scarcely cause environmental pollution since the soaps are bound to Ca and Mg to precipitate as metal soaps, which microorganisms feed on in the environment.
(5) The soap is made of natural materials and exhibits its effect in a trace amount, and thus may be used worry-free for foods, tableware, underwear, and baby goods, etc.
(6) When the number of unsaturated bonds is increased such as linoleic acid (C18:2) and linolenic acid (C18:3), the product is easily oxidized and deteriorates. The C18 unsaturated fatty acid soap composed mainly of the oleic acid (C18:1) is difficult to deteriorate and is excellent in stability.
(7) The percentage of the C18 unsaturated fatty acid soap in the surface-active agent is 20 to 100% by weight and more preferably 30 to 100% by weight. Thus, when used by diluting to 0.1 to 3% by weight or less with water, excellent antiviral performance is exhibited, and neither a foreign body sensation nor irritation is felt on the skin even if the antiviral agent is not wiped off after use.
(8) The oleate soap is abundantly contained in an amount of 50% by weight or more. Thus, it is possible to use as a liquid cleansing agent that gives less irritation to the skin and has a good use feeling such as a good moisturizing feeling, excellent foam quality and a foam life, as well as an excellent oxidation stability. This cleansing agent not only rinses off a virus physically, but also has high inactivation ability against infectivity of a virus such as an influenza virus and a norovirus and is excellent in germicidal properties.
(9) By making the summed amount of the laurate soap and the myristate soap 70% by weight or more, and making the percentage of the palmitate soap and the percentage of the stearate soap less than 15% by weight based on the total amount of the saturated fatty acid soaps, it is possible to enhance the foaming property and the foam quality and further reduce the viscosity at low temperature.

Here, the C18 unsaturated alkyl group may include an oleyl group (C18:1), a linoleyl group (C18:2), and a linolenyl group (C18:3). Of those, the oleyl group is suitable since it is more difficult to deteriorate and is more excellent in stability than the linoleyl group and the linolenyl group.

As the surface-active agent having the C18 unsaturated alkyl group, the surface-active agents used for synthetic cleansers and cosmetics, for example, sulfonate ester, sulfate salts, phosphate salts and sarcosine salts, etc., in addition to so-called soaps such as sodium salts, potassium salts, ammonium salts, arginine salts, alkanol amine salts such as triethanol ammonium salts or composite salts thereof of fatty acids are used, and may be used alone or in combination of two or more. The concentration of the surface-active agent having the C18 unsaturated alkyl group when using the antiviral agent, is suitably 0.1% by weight or more. When the concentration is less than 0.1% by weight, the antiviral performance is not obtained sufficiently. When the concentration is more than 3% by weight, although a cleansing ability appears, irritation and stickiness are felt on the skin when left on the skin, and it becomes necessary to wipe it off or rinse it off with water.

It is also possible to combine the surface-active agent having the C18 unsaturated alkyl group with another surface-active agent, antimicrobial compound or antiviral compound. This may favorably enhance the effect of the antimicrobial compound or the antiviral compound by a synergistic effect.

Here, the C18 unsaturated fatty acid may include an oleic acid (C18:1), a linoleic acid (C18:2), and a linolenic acid (C18:3). Of those, the oleic acid (C18:1) is suitable since the oleic acid (C18:1) is more difficult to deteriorate and is more excellent in stability than the linoleic acid and the linolenic acid.

As the C18 unsaturated fatty acid soap, sodium salts, potassium salts, ammonium salts, arginine salts, alkanol amine salts such as triethanol ammonium salts or composite salts thereof of the fatty acids are used, and may be used alone or in combination of two or more.

The antiviral agent of the present invention may be used in various modes, e.g., as a liquid cleaning agent or a disinfectant that cleans the hands and face, etc., as a wiper or a mask by being impregnated in cloth, etc., as a disinfectant used for a foot washing bath and a foot cleaning mat, etc., or as an air spray for spraying to a subject. The concentration of the C18 unsaturated fatty acid soap when using the antiviral agent is suitably 0.1% by weight or more. When the concentration is less than 0.1% by weight, the antiviral performance is not obtained sufficiently. When the concentration is more than 3% by weight, although the cleansing ability appears, irritation and stickiness are felt on the skin when left on the skin, and it becomes necessary to wipe it off or rinse it off with water.

Here, when the percentage of the C18 unsaturated fatty acid soap in the surface-active agent is less than 30% by weight, if used at a concentration at which the antiviral performance is obtained, sometimes irritation is felt or redness occurs on the skin, and wiping off or rinsing off with water becomes necessary after use. When the antiviral agent is diluted to the concentration at which neither the foreign body sensation such as stickiness nor irritation is felt on the skin, the antiviral performance is low and its effect becomes unclear. When the percentage is less than 20% by weight, the tendency has a pronounced effect and it is not preferable.

The invention according to a second aspect of the present invention is the antiviral agent according to the first aspect, and has the constitution in which a percentage of the soaps in the antiviral agent is 0.5 to 40% by weight.

The following action in addition to the actions of the first aspect is obtained by this constitution.

(1) The percentage of the soaps in the antiviral agent is 0.5 to 40% by weight. Thus, handleability and foaming property are excellent.

The invention according to a third aspect of the present invention is the antiviral agent according to the first or second aspect, and has the constitution in which the soap that is any one of a potassium salt, a sodium salt, an arginine salt, an ammonium salt, and a triethanolamine salt of the fatty acid is the major component.

The following action in addition to the actions of the first or second aspect is obtained by this constitution.

(1) A potassium fatty acid soap, a sodium fatty acid soap, an arginine fatty acid soap, an ammonium fatty acid soap, and a triethanolamine fatty acid soap are bound to calcium and magnesium to become metal soaps in the environment, rapidly lose an interfacial activation effect, and lose toxicity. Underwater organisms feed on the metal soap, and thus the soap is highly biodegradable and has a low environmental load.

The invention according to a fourth aspect of the present invention is the antiviral agent according to any one of the first to the third aspects, and has the constitution in which an inactivation ability against viral infectivity of an influenza virus is high.

The same actions as in the first to the third aspects are obtained by this constitution.

The invention according to a fifth aspect of the present invention is the antiviral agent according to any one of the first to the third aspects, and has the constitution in which an inactivation ability against viral infectivity of a feline calicivirus is high.

The same actions as in the first to the third aspects are obtained by this constitution.

The invention according to a sixth aspect of the present invention has the constitution including the antiviral agent according to any one of the first to fifth aspects.

The following actions are obtained by this constitution.

(1) Even when the cleansing agent is diluted with water, excellent antiviral activity is obtained in the typical concentration range for cleansing since the oleate soap that is the active component exhibiting the antiviral activity is abundantly contained. The oleate soap exhibits the antiviral activity at the concentration at which the lather is not obtained by being diluted with water. Therefore, in the process of hand-washing, higher antiviral activity is obtained than the activity obtained by rinsing off by the ordinary cleansing agent, and it is possible to prevent the expansion of viral contamination.
(2) By making the summed amount of the laurate soap and the myristate soap 70% by weight or more, and making the percentage of the palmitate soap and the percentage of the stearate soap less than 15% by weight based on the total amount of the saturated fatty acid soaps, it is possible to enhance the foaming property and the foam quality and further reduce the viscosity at low temperature.
(3) The cleansing agent exerts an excellent cleansing power regardless of water temperature, and is excellent in oxidation stability.

Here, when the oleate soap is derived from natural fats and oils, other soap components are mixed in, but it suffices if the percentage of the oleate soap is 50% by weight or more. The fatty acid that composes the soap is selected from straight or branched chain saturated fatty acids or unsaturated fatty acid having about 12 to 22 carbon numbers, and preferably about 12 to 18 carbon numbers. For example, a C12 saturated fatty acid, a C14 saturated fatty acid, a C16 saturated fatty acid, a C16 unsaturated fatty acid and a C18 saturated fatty acid may include a lauric acid, a myristic acid, a palmitic acid, a palmitoleic acid and a stearic acid, respectively. The C18 unsaturated fatty acids may include an oleic acid, a linoleic acid and a linolenic acid. The unsaturated fatty acids such as an arachidonic acid (C20), a cetoleic acid (C22), an erucic acid (C22) and a brassidic acid (C22) may be included.

The antiviral agent and the cleansing agent of the present invention may combine perfumes, coloring agents, fluorescent brightening agents, various vitamins, plant extracts, surface-active agents other than the soap, antioxidants, preservatives, alcohols, sugars, thickeners, water-soluble polymers, fats and oils including essential oil, etc., moisturizing agents, and additionally, antimicrobial compounds and antiviral compounds, etc., in addition to the soap and water. In this regard, however, it is preferable not to combine the surface-active agent other than the soap and the additives such as the antioxidant, the antimicrobial compound and the antiviral compound, in terms of safety such as reduction of cytotoxicity for the skin as much as possible, in terms of protection of the natural environment by reducing the environmental load as much as possible, and in terms of enhancing the antiviral effect as much as possible.

Considering the handleability and foaming property, etc., the concentration of the soap in the antiviral agent and the cleansing agent of the present invention is suitably 0.5 to 40% by weight.

The soap in the antiviral agent and the cleansing agent of the present invention may be produced directly from the fats and oils by a saponification method. The soap may also be produced by the fatty acid reacting with alkali (neutralization method). The soap obtained by the saponification method is suitable since the cytotoxicity may further be reduced and the moisturizing property for the skin is good due to the soap containing impurities such as glycerin and the like.

The fats and oils are not particularly limited, and may include soybean oil, corn oil, rice bran oil, rapeseed oil, cotton seed oil, coconut oil, palm kernel oil, palm oil, lard, fish oil, beef tallow, olive oil, camellia oil, castor oil, linseed oil, sunflower oil, earthnut oil, sesame oil, nut oil, peanut oil, grape seed oil, safflower oil, avocado oil, rice oil, cacao butter, and shea butter, etc.

When produced by the saponification method, the fatty acid soap having an appropriate percentage composition may be obtained by mixing multiple kinds of fats and oils to make a raw material fat and oil.

When produced by the neutralization method, the fatty acid soap may be used in the same manner as the soap obtained by the saponification method, by adding glycerin. This method is advantageous in that a stable quality is always obtained by using the purified fatty acid to be combined.

The soap obtained by the saponification method and the soap obtained by the neutralization method may be mixed and used. This is advantageous in that the range of the raw material that may be used is expanded by adjusting the composition of the soap derived from the natural fats and oils and obtained by the saponification method with the soap obtained by the neutralization method.

Here, according to the findings of the inventors of the present invention, as the summed amount of the laurate soap and the myristate soap is less than 70% by weight based on the total amount of the saturated fatty acid soaps, the foaming property is reduced, which is not preferable. Also, the stability tends to be reduced, e.g., white turbidity or precipitation occurs when a product such as a hand soap in which the soap is dissolved in a liquid is produced. Thus, this is not preferable. When the percentage of the palmitate soap and the percentage of the stearate soap exceed 15% by weight based on the total amount of the saturated fatty acid soaps, the soap becomes difficult to dissolve, and even if dissolved, the stability is reduced, e.g., white turbidity or the precipitation occurs. Thus, this is also not preferable. When the amount of the oleate soap exceeds 75% by weight based on the total amount of the soaps, the nature of the oleate soap is strongly expressed, and thus no disadvantage due to these unsaturated fatty acid soaps is seen.

### Advantageous Effects of Invention

As described above, according to the antiviral agent and the cleansing agent of the present invention, advantageous effects are obtained as follows.

According to the invention described in the first aspect,

(1) it is possible to provide the antiviral agent that is excellent in inactivation ability against infectivity of a virus such as an influenza virus and a norovirus since the antiviral agent contains the surface-active agent having the C18 unsaturated alkyl group as the active component.
(2) It is possible to provide the antiviral agent that does not require being lathered or rinsed off with water like cleansing agents since the antiviral agent may inactivate a virus at a very low concentration.
(3) It is possible to provide the antiviral agent that scarcely causes environmental pollution since different from the synthetic surface-active agent, the soap is bound to Ca and Mg to become a metal soap and precipitate in the environment, which microorganisms feed on.
(4) It is possible to provide the antiviral agent that may be used worry-free for foods, tableware, underwear, and baby goods, etc., since the antiviral agent is made of natural materials and exhibits the effect in a trace amount.
(5) It is possible to provide the antiviral agent that is difficult to deteriorate and is excellent in stability since the C18 unsaturated fatty acid soap is composed mainly of the oleic acid (C18:1) whereas the product is easily oxidized and deteriorates when the number of the unsaturated bonds increases such as the linoleic acid (C18:2) and the linolenic acid (C18:3).
(6) It is possible to provide the antiviral agent that exhibits excellent antiviral performance when used by being diluted and neither a foreign body sensation nor irritation to the skin is felt even if the antiviral agent is not wiped off after use since the percentage of the C18 unsaturated fatty acid soap in the surface-active agent is 20 to 100% by weight.
(7) The oleate soap is abundantly contained. Thus, it is possible to provide the antiviral agent that may be used as the liquid cleansing agent that gives less irritation to the skin and has a good use feeling such as a good moisturizing feeling, excellent foaming property and foam life as well as an excellent oxidation stability, and that not only rinses off a virus physically but also has high inactivation ability against infectivity of a virus such as an influenza virus and a norovirus and is excellent in germicidal properties.
(8) It is possible to provide the antiviral agent that not only is excellent in inactivation ability against a viral infection but also enhances the foaming property and the foam quality, further may reduce the viscosity at low temperature and keep a liquid form even at low temperature, by making the summed amount of the laurate soap and the myristate soap 70 to 100% by weight, and making the percentage of the palmitate soap and the percentage of the stearate soap less than 15% by weight based on the total amount of the saturated fatty acid soaps.

According to the invention described in the second aspect, in addition to the effects in the first aspect,

(1) it is possible to provide the antiviral agent having excellent handleability and foaming property.

According to the invention described in the third aspect, in addition to the effects in the first or second aspect,

(1) it is possible to provide the antiviral agent that is highly biodegradable and has a low environmental load since the potassium fatty acid soap, the sodium fatty acid soap, the arginine fatty acid soap, the ammonium fatty acid soap, and the triethanolamine fatty acid soap are bound to calcium and magnesium to become metal soaps in the environment, lose the interfacial activation effect, and lose toxicity, and underwater organisms feed on the metal soap.

According to the invention described in the fourth aspect, the same effects as in the first to the third aspects are obtained.

According to the invention described in the fifth aspect, the same effects as in the first to the third aspects are obtained.

According to the invention described in the sixth aspect,

(1) it is possible to provide the cleansing agent that exhibits higher antiviral activity than the activity obtained by rinsing off by the ordinary cleansing agent in the process of the hand-washing and may prevent the expansion of viral contamination since the oleate soap that is the active component exhibiting the antiviral activity is abundantly contained and thus, even when the cleansing agent is diluted with water, excellent antiviral activity is obtained in the typical concentration range for cleansing, and since the oleate soap exhibits the antiviral activity at a concentration at which the lather is not obtained by being diluted with water.
(2) It is possible to provide the cleansing agent that not only is excellent in inactivation ability against a viral infection but also enhances the foaming property and the foam quality, further may reduce the viscosity at low temperature and keep a liquid form even at low temperature, by making the summed amount of the laurate soap and the myristate soap 70 to 100% by weight, and making the percentage of the palmitate soap and the percentage of the stearate soap less than 15% by weight based on the total amount of the saturated fatty acid soaps.
(3) It is possible to provide the cleansing agent that exerts excellent cleansing power regardless of the water temperature and is excellent in oxidation stability and antiviral activity.
(4) It is possible to provide the cleansing agent that may be used as the liquid cleansing agent that gives less irritation to the skin and has a good use feeling such as a good moisturizing feeling, excellent foaming property and foam life as well as deterioration stability, and that not only rinses off a virus physically but also has high inactivation ability against infectivity of a virus such as an influenza virus and a norovirus and is excellent in germicidal properties.

### BRIEF DESCRIPTION OF DRAWINGS

FIG. 1 is a graph showing an inactivation ability of an antiviral agent against a feline calicivirus (Example 1).

FIG. 2 is a graph showing an inactivation ability of an antiviral agent against an avian influenza virus (Example 2).

FIG. 3 is a graph showing the inactivation ability of an antiviral agent of a sodium oleate soap and a potassium oleate soap against the avian influenza virus (Example 4).

FIG. 4 is a graph showing the inactivation ability against the feline calicivirus when a potassium saturated fatty acid soap having a carbon chain different in length was mixed with a potassium C18:1 fatty acid soap (Example 5).

FIG. 5 is a graph showing effects of diluted concentrations on the inactivation ability of an antiviral agent, sample No. 19 against the feline calicivirus (Example 6).

FIG. 6 is a graph showing the effects of diluted concentrations on the inactivation ability of the antiviral agent, sample No. 19 against the avian influenza virus (Example 6).

FIG. 7 is a graph comparing the inactivation ability against the feline calicivirus when the antiviral agent was the sample No. 19 alone and when various additives were added thereto (Example 7).

FIG. 8 is a graph comparing the inactivation ability against the avian influenza virus when the antiviral agent was the sample No. 19 alone and when various additives were added thereto (Example 7).

FIG. 9 is a graph comparing the inactivation ability of surface-active agents other than the soap and the antiviral agent, sample No. 19 against the feline calicivirus (Example 8).

FIG. 10 is a graph comparing the inactivation ability of surface-active agents other than the soap and the antiviral agent, sample No. 19 against the avian influenza virus (Example 8).

FIG. 11 is a graph comparing the inactivation ability of synthetic surface-active agents having a C18 unsaturated alkyl chain and the potassium oleate soap (C18:1) against the feline calicivirus (Example 9).

FIG. 12 is a graph comparing the inactivation ability of the synthetic surface-active agents having the C18 unsaturated alkyl chain and the potassium oleate soap (C18:1) against the avian influenza virus (Example 9).

FIG. 13 is a graph comparing the inactivation ability against the feline calicivirus in potassium saturated fatty acid soaps having the carbon chain different in length and potassium fatty acid soaps different in C18 unsaturation degree (Example 10).

FIG. 14 is a graph comparing the inactivation ability against the avian influenza virus in potassium saturated fatty acid soaps having the carbon chain different in length and potassium fatty acid soaps different in C18 unsaturation degree (Example 10).

FIG. 15 is a graph showing the inactivation ability of the antiviral agent, sample No. 19 against a new influenza virus (Example 11).

### DESCRIPTION OF EMBODIMENTS

### Embodiment 1

Embodiments for carrying out the present invention will be described below. The present invention is not limited to these embodiments.

The antiviral agent according to the present invention is provided in a concentrated liquid form or a powder form. A user dilutes this with water or hot water for use. The antiviral agent was dissolved in water at room temperature and actually examined at various concentrations, and the concentration of the surface-active agent having the C18 unsaturated alkyl group during use was 0.1 to 3% by weight and more preferably 0.3 to 1% by weight. When the concentration of the surface-active agent having the C18 unsaturated alkyl group is less than 0.3% by weight, the antiviral effect is reduced, and when it is less than 0.1% by weight, the antiviral effect is remarkably reduced. Thus, this is not preferable. When the concentration exceeds 3% by weight, the antiviral agent exhibits the lather and cleansing effect, but stickiness or a foreign body sensation is felt or redness occurs on the skin unless the antiviral agent is wiped off or rinsed off with water. Thus, this is also not preferable. When the antiviral agent is allowed to act upon a subject by dripping this diluted solution on hands or feet and rubbing it, wiping with diluted solution impregnated in cloth, etc., or spraying the diluted solution with an atomizer, the antiviral agent acts upon the virus to inactivate it in a short period of time. Thus, subsequently, the antiviral agent may be wiped off with a cloth or rinsed off with water as needed. The foam life is very short at this concentration, and the antiviral agent had no cleansing ability as the surface-active agent.

### Embodiment 2

Water or hot water is added to a medicinal solution vessel having a size in which the subject to be treated with antiviral agent may be immersed, and the antiviral agent of the present invention is added thereto and dissolved by stirring so that the concentration of the surface-active agent having the C18 unsaturated alkyl group is 0.1 to 3% by weight and more preferably 0.5 to 1% by weight. The virus on the surface of the subject to be treated is inactivated by immersing the subject in this medicinal solution vessel. The influenza virus, etc., on a petri dish could be inactivated immediately by immersing the petri dish. The same method may be applied to hands and fingers and tableware. As a modified method thereof, the antiviral agent may be utilized by adding and dissolving it in a foot washing bath for domestic animals, etc. The antiviral agent acts in a short period of time, and inactivates the virus. Thus, a step of rinsing with water or wiping off may be provided after the treatment in the medicinal solution vessel and the foot washing bath.

### Embodiment 3

The shape and the use method of the cleansing agent according to the present invention are the same as those of conventional body shampoos, hand soaps and medicinal soaps. By using in the same manner as with the conventional products, the virus is inactivated during the washing and the prevalence of the viral infection may be prevented more effectively, in addition to the physical washing off effect of the conventional cleansing agents on the virus.

The present invention will be described more specifically with reference to the following Examples, but the present invention is not limited thereto.

### Example 1

### Preparation of Potassium Fatty Acid Soap

A lauric acid (NAA-122 manufactured by NOF Corporation), a myristic acid (NAA-142 manufactured by NOF Corporation), a palmitic acid (NAA-160 manufactured by NOF Corporation), a stearic acid (NAA-180 manufactured by NOF Corporation), and an oleic acid (EXO-S manufactured by NOF Corporation) were mixed at a weight ratio shown in Table 1.

0.175 M of potassium hydroxide was dissolved in 300 mL of purified water, heated up to 60 to 70° C., and the fatty acids corresponding to 0.175 M mixed according to Table 1 were added thereto and mixed well. The mixture was adjusted to 500 mL with purified water, and gradually cooled to room temperature to obtain 0.35 M (molar concentration of soap total amount) of an antiviral agent (sample Nos. 1 to 18).

TABLE 1

| Fatty | Sample | | | | | | | | | | | | | | | | | | |
|---|---|---|---|---|---|---|---|---|---|---|---|---|---|---|---|---|---|---|---|
| acid | No. | 1 | 2 | 3 | 4 | 5 | 6 | 7 | 8 | 9 | 10 | 11 | 12 | 13 | 14 | 15 | 16 | 17 | 18 |
| Lauric | C12 | 15 | 60 | 37.5 | 50 | 50 | 50 | 10 | 40 | 25 | 30 | 30 | 30 | 7.5 | 17.5 | 12.5 | 12.5 | 12.5 | 12.5 |
| acid | | | | | | | | | | | | | | | | | | | |
| Myristic | C14 | 60 | 15 | 37.5 | 10 | 10 | 10 | 40 | 10 | 25 | 10 | 10 | 10 | 17.5 | 7.5 | 12.5 | 5 | 5 | 5 |
| acid | | | | | | | | | | | | | | | | | | | |
| Palmitic | C16 | 0 | 0 | 0 | 5 | 12.5 | 15 | 0 | 0 | 0 | 2.5 | 7.5 | 10 | 0 | 0 | 0 | 2.5 | 5 | 7.5 |
| acid | | | | | | | | | | | | | | | | | | | |
| Stearic | C18 | 0 | 0 | 0 | 10 | 2.5 | 0 | 0 | 0 | 0 | 7.5 | 2.5 | 0 | 0 | 0 | 0 | 5 | 2.5 | 0 |
| acid | | | | | | | | | | | | | | | | | | | |
| Oleic | C18:1 | 25 | 25 | 25 | 25 | 25 | 25 | 50 | 50 | 50 | 50 | 50 | 50 | 75 | 75 | 75 | 75 | 75 | 75 |
| acid | | | | | | | | | | | | | | | | | | | |
| | Total | 100 | 100 | 100 | 100 | 100 | 100 | 100 | 100 | 100 | 100 | 100 | 100 | 100 | 100 | 100 | 100 | 100 | 100 |

(%)

### (Measurement of Inactivation Ability Against Infectivity of Feline Calicivirus)

It is impossible to culture a norovirus in cells. Thus, it is common to analogize using feline calicivirus that is a related virus of a norovirus when the infectivity of a norovirus is evaluated. Thus, the inactivation ability of the antiviral agents (sample Nos. 1 to 18) in Example 1 against infectivity of the feline calicivirus was examined.

The feline calicivirus (family: Caliciviridae, genus: vesivirus) as the virus, CRFK cells (derived from feline kidney) as the cell, and Eagle's minimum essential medium (MEM) as a diluent, and penicillin G and streptomycin were used.

First, in order to exclude the effect of the serum included in a virus solution, the virus solution was previously diluted to 10 times with the diluent. 10 μL of this diluted virus solution and 90 μL of the antiviral agent (sample Nos. 1 to 18) diluted to 100 times were mixed, and reacted at room temperature for 3 minutes. Subsequently, the reaction mixture was diluted with the diluent to prepare a serial dilution of 10 times.

A monolayer of CRFK cells in a 96-well plate was washed once with phosphate buffered saline (PBS), and the diluted virus solution was inoculated therein (50 μL/well). The plate was incubated for adsorption and penetration of the virus in a carbon dioxide incubator for one hour, subsequently washed once with PBS, and the cell maintenance solution (same as the diluent) was added (100 μL/well) to culture the cells. When a cytopathic effect (CPE) extended after the 4th day, the cells were fixed and stained. Using the Behrens-Kaerber method, a 50% infection dose was evaluated, and a virus infection dose (unit: 50% tissue culture infectious dose [TCID50]) (/mL) was calculated.

For comparison, PBS was used in place of the antiviral agent, the same experiment was carried out, and the corresponding virus infection dose was calculated.

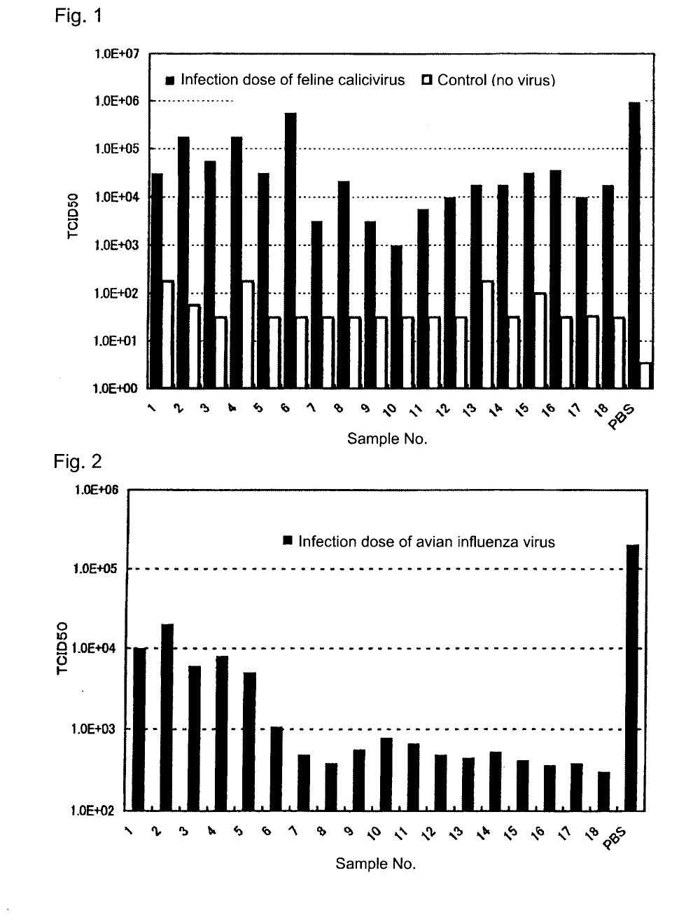

The inactivation ability of the antiviral agents of the sample Nos. 1 to 18 shown in Table 1 in Example 1 against the feline calicivirus is shown in FIG. 1. A solid bar denotes the infection dose after the feline calicivirus was added, and it is shown that the lower the dose is, the higher the inactivation ability is. The bar on the extreme right shows the case of using PBS, and indicates the infection dose of the virus stock solution before being inactivated. An open bar shows an apparent infection dose measured when the feline calicivirus was not inoculated. Because of cytotoxicity of the reagent itself, the sample appears to have the infection dose although the virus was not inoculated. It is shown that the higher this value is, the more frequently the cytotoxicity occurs in the sample.

The virus infection dose was decreased to about one-hundredth in the sample Nos. 7 to 18 that contained oleate (C18:1) soap in an amount of 50% by weight or more, while the virus infection dose was decreased to one-tenth in the sample Nos. 1 to 6 that contained oleate soap in an amount of 25% by weight. Thus, it was shown that it was preferable that the concentration of the oleate soap was 50% by weight or more when the feline calicivirus was inactivated at a concentration of 0.0035 M. It was also shown that the cytotoxicity was low in any of the antiviral agents of the sample Nos. 1 to 18.

### Example 2

### Measurement of Inactivation Ability Against Infectivity of Avian Influenza Virus

The inactivation ability of the antiviral agents (sample Nos. 1 to 18) prepared in Example 1 against the avian influenza virus was examined.

Influenza virus A/whistling swan/Shimane/499/83 (H₅N3) (family: Orthomyxoviridae, genus: influenza virus A) as the virus, MDCK (+) cells as the cell, and Dulbecco's modified Eagle's minimum essential medium (DMEM) supplemented with penicillin G, streptomycin, amphotericin B and crude trypsin as the diluent were used.

10 μL of the virus solution and 90 μL of the antiviral agent (sample Nos. 1 to 18) diluted to 100 times were mixed, and reacted at room temperature for 3 minutes. Subsequently, the reaction mixture was diluted with the diluent (DMEM) to make a serial dilution of 10 times. The diluted virus solution was inoculated to a monolayer cell culture in a 96-well plate (100 μL/well), and the cells were cultured. When the cytopathic effect (CPE) extended after the 4th day, the cells were fixed and stained. Using the Behrens-Kaerber method, a 50% infection dose was evaluated, and the virus infection dose (unit: 50% tissue culture infectious dose [TCID50]) (/mL) was calculated.

For comparison, the phosphate buffered saline (PBS) was used in place of the antiviral agent, the same experiment was carried out, and the corresponding virus infection dose was calculated.

The results of measuring the inactivation ability against the avian influenza virus (Example 2) are shown in FIG. 2. The solid bar denotes the infection dose. It is shown that the lower the infection dose is, the higher the inactivation ability is. The bar on the extreme right shows the case of using PBS, and indicates the infection dose of the virus stock solution before being inactivated.

The virus infection dose was decreased closely to about one-thousandth in the sample Nos. 7 to 18 that contained oleate (C18:1) in an amount of 50% by weight or more, while the virus infection dose was decreased to about one-hundredth in the sample Nos. 1 to 6 that contained oleate in an amount of 25% by weight. The antiviral agents showed the higher inactivation ability against the avian influenza virus than against the feline calicivirus. The cause is thought to be a difference in sensitivity to the antiviral agents that the feline calicivirus has no envelope whereas the avian influenza virus has the envelope. From these results, it was shown that it was preferable that the concentration of the oleate was 25% by weight or more and more preferably 50% by weight or more when the avian influenza virus was inactivated at a concentration of 0.0035 M.

### Example 3

### Evaluation of Foaming Property, Cleansing Property and Use Feeling

A foaming property, a foam life, a cleansing property, a use feeling (moisturizing feeling), and stability were evaluated when the antiviral agents (sample Nos. 1 to 18) prepared in Example 1 were used as the cleansing agents.

A sensory evaluation for the evaluation parameters was performed by three panelists according to the following criteria, and an average of their scores was obtained.

For the foaming property, when 0.3 g of each antiviral agent was jetted from a pump foamer and spread on hands, when a sufficient amount of foam was created, a score of 5 was recorded, when a small amount of foam was created, a score of 3 was recorded, and when hardly any foam was created, a score of 1 was recorded (intermediate scores were 4 and 2, respectively).

For the foam life, when the foam created in the evaluation of the foaming property was kept for one minute or more, a score of 5 was recorded, when the foam was slightly decreased, a score of 3 was recorded, and when the foam almost disappeared, a score 1 was recorded (intermediate scores were 4 and 2, respectively).

For the cleansing property, when female panelists with oily skin washed their face using 1 g of the antiviral agent, when there was no stickiness on the forehead and nasal bridge after washing the face, which is a site with a large amount of sebum, and a refreshing feeling was obtained, a score of 5 was recorded, when there was some stickiness, a score of 3 was recorded, and when there was a clear stickiness, a score 1 was recorded (intermediate scores were 4 and 2, respectively).

For the use feeling, after washing the face for evaluating the cleansing property, when there was a moisturizing feeling, a score of 5 was recorded, when there was some moisturizing feeling, a score of 3 was recorded, and when there was no moisturizing feeling, a score 1 was recorded (intermediate scores were 4 and 2, respectively).

For the stability, when the 0.35 M solution placed in an incubator at 1° C. was precipitated or produced white turbidity, a score of 1 was recorded, and when there was no change with clearness, a score of 5 was recorded.

TABLE 2

| | Panelist 1 | | | | | Panelist 2 | | | | | Panelist 3 | | | | | |
|---|---|---|---|---|---|---|---|---|---|---|---|---|---|---|---|---|
| | Sensory test | | | | | Sensory test | | | | | Sensory test | | | | | |
| | Foam- | | Cleann- | | | Foam- | | Cleann- | | | Foam- | | Cleann- | | | Aver- |
| | ing | Foam- | ing | Use | Sta- | ing | Foam- | ing | Use | Sta- | ing | Foam- | ing | Use | Sta- | age |
| No. | property | life | property | feeling | bility | property | life | property | feeling | bility | property | life | property | feeling | bility | value |
| 1 | 4 | 2 | 3 | 2 | 1 | 4 | 2 | 4 | 4 | 1 | 4 | 2 | 3 | 4 | 1 | 13.7 |
| 2 | 3 | 3 | 3 | 3 | 5 | 3 | 4 | 3 | 4 | 5 | 3 | 4 | 3 | 3 | 5 | 18.0 |
| 3 | 5 | 5 | 4 | 4 | 1 | 5 | 4 | 3 | 5 | 1 | 4 | 3 | 2 | 2 | 1 | 16.3 |
| 4 | 4 | 5 | 5 | 2 | 1 | 4 | 2 | 3 | 4 | 1 | 5 | 5 | 3 | 5 | 1 | 16.7 |
| 5 | 4 | 5 | 3 | 4 | 1 | 4 | 3 | 4 | 4 | 1 | 4 | 4 | 4 | 4 | 1 | 16.7 |
| 6 | 5 | 3 | 3 | 3 | 1 | 4 | 4 | 4 | 3 | 1 | 4 | 5 | 4 | 3 | 1 | 16.0 |
| 7 | 5 | 5 | 5 | 2 | 1 | 4 | 4 | 3 | 3 | 1 | 4 | 4 | 3 | 3 | 1 | 16.0 |
| 8 | 3 | 5 | 4 | 2 | 5 | 4 | 3 | 4 | 4 | 5 | 4 | 3 | 4 | 4 | 5 | 19.7 |
| 9 | 3 | 2 | 4 | 2 | 5 | 5 | 5 | 3 | 4 | 5 | 5 | 5 | 3 | 4 | 5 | 20.0 |
| 10 | 4 | 3 | 3 | 3 | 1 | 5 | 4 | 3 | 3 | 1 | 4 | 4 | 3 | 3 | 1 | 15.0 |
| 11 | 3 | 2 | 4 | 4 | 5 | 3 | 2 | 3 | 4 | 5 | 4 | 2 | 4 | 3 | 5 | 17.7 |
| 12 | 3 | 5 | 4 | 4 | 1 | 5 | 4 | 3 | 4 | 1 | 4 | 4 | 3 | 4 | 1 | 16.7 |
| 13 | 2 | 3 | 4 | 3 | 5 | 3 | 4 | 3 | 4 | 5 | 3 | 4 | 3 | 4 | 5 | 18.3 |
| 14 | 2 | 3 | 4 | 4 | 5 | 3 | 2 | 3 | 4 | 5 | 3 | 2 | 3 | 4 | 5 | 17.3 |
| 15 | 1 | 3 | 4 | 3 | 5 | 2 | 2 | 3 | 4 | 5 | 2 | 2 | 3 | 4 | 5 | 16.0 |
| 16 | 3 | 3 | 4 | 4 | 1 | 4 | 4 | 3 | 4 | 1 | 3 | 4 | 4 | 3 | 1 | 15.3 |
| 17 | 1 | 4 | 4 | 4 | 5 | 2 | 2 | 4 | 4 | 5 | 2 | 3 | 4 | 4 | 5 | 17.7 |
| 18 | 2 | 3 | 4 | 4 | 5 | 3 | 2 | 3 | 4 | 5 | 3 | 3 | 5 | 4 | 5 | 18.3 |

5 Good
4 Slightly good
3 Ordinary
2 Slightly bad
1 Bad

The results in Example 3 are shown in Table 2. When the amount of the oleate soap was less than 50% by weight based on the total amount of the soaps (sample Nos. 1 to 6) when used as the cleansing agent, the average score of the summed evaluations generally tended to be low.

Even when the oleate soap was contained in an amount of 50% by weight or more based on the total amount of the soaps (sample Nos. 7 to 18), if the palmitate soap or the stearate soap in the saturated fatty acids was contained in an amount of 15% by weight or more (sample Nos. 10 and 16), the average score of the summed evaluations was low. From these results, it was shown that the amount of the palmitate soap or the stearate soap was preferably less than 15% by weight when the amount of the oleate soap was 50 to 75% by weight based on the total amount of the soaps.

### Example 4

### Comparison Between Sodium Oleate and Potassium Oleate

A potassium oleate soap (0.35 M) was prepared in the same manner as in Example 1, except that 0.175 M of oleic acid was used. Likewise, a sodium oleate soap (0.35 M) was prepared in the same manner as above, except that 0.175 M of sodium hydroxide was used in place of 0.175 M of potassium hydroxide.

Using these oleate soaps as the antiviral agents, the inactivation ability against the avian influenza virus was measured in the same manner as in Example 2.

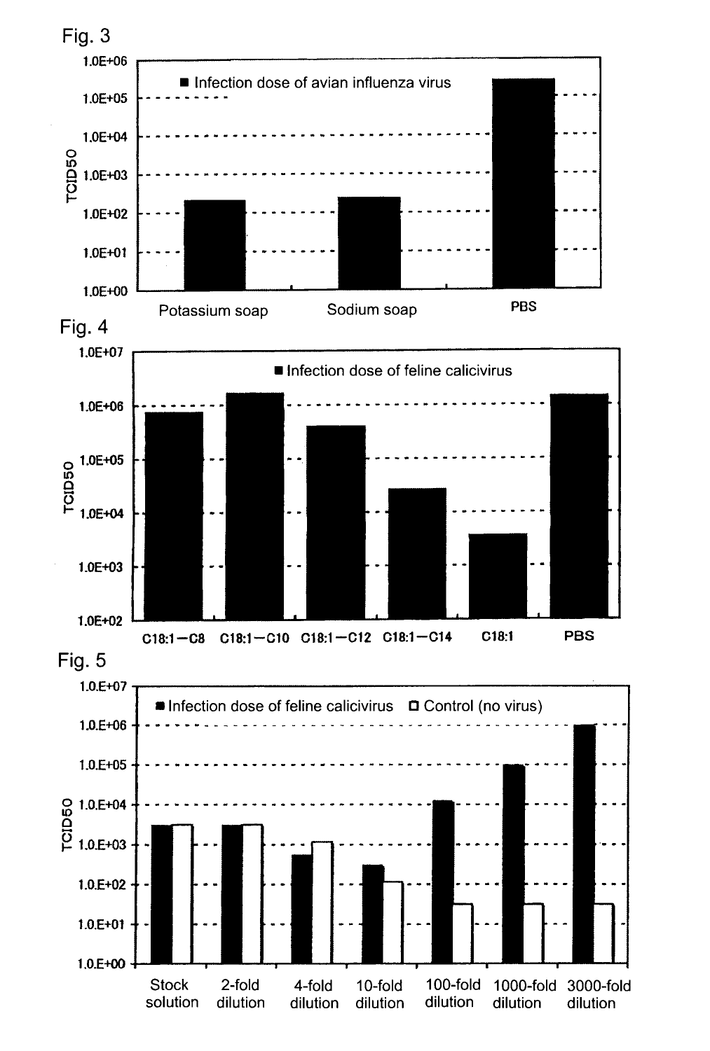

The results in Example 4 are shown in FIG. 3. There was no difference in the ability to inactivate the avian influenza virus between the potassium oleate soap and the sodium oleate soap. Likewise, there was no difference in the inactivation ability against the feline calicivirus between the potassium oleate soap and the sodium oleate soap.

### Example 5

### Examination of Mixture of Oleate Soap and Saturated Fatty Acid Soap

A 0.35 M solution of potassium soap of each fatty acid was prepared in the same manner as in Example 1, for oleic acid alone, caprylic acid (NAA-82 manufactured by NOF Corporation) alone, capric acid (NAA-102 manufactured by NOF Corporation) alone, lauric acid alone, and myristic acid alone.

Seven parts by volume of the potassium soap of the other fatty acid was mixed with 3 parts by volume of this potassium oleate soap to use as the antiviral agent (total soap concentration was 0.35 M).

Using these antiviral agents, the inactivation ability against the avian influenza virus was measured in the same manner as in Example 2 (the antiviral agent was diluted to 100 times and mixed with the virus, thus the soap concentration was 0.0035 M in the measurement).

The results in Example 5 are shown in FIG. 4. The inactivation ability was scarcely exhibited when the caprylate soap (C8), the caprate soap (C10) and the laurate soap (C12) were used. The myristate soap (C14) having a longer fatty acid chain than the lauric acid exhibited the inactivation ability close to when the oleate soap (C18:1) alone was used. It was suggested that the length of the fatty acid chain was involved in the inactivation of the virus.

### Example 6

### Preparation of Fatty Acid Mixed Antiviral Agent

An antiviral agent (total soap concentration was 0.35 M) (sample No. 19) was prepared in the same manner as in Example 1, except that the lauric acid, the myristic acid, the palmitic acid, the stearic acid and the oleic acid were mixed at a molar ratio of 25:9:1:0:65.

### (Effect of Concentration of Fatty Acid Mixed Antiviral Agent on Inactivation Ability Against Virus)

The antiviral agent of the sample No. 19 was diluted with purified water. Using this antiviral agent with various concentrations, the effect of the concentration of the antiviral agent (sample No. 19) on the ability to inactivate the feline calicivirus was examined in the same manner as in Example 1.

Also, the effect of the concentration of the antiviral agent (sample No. 19) on the ability to inactivate the avian influenza virus was examined in the same manner as in Example 2.

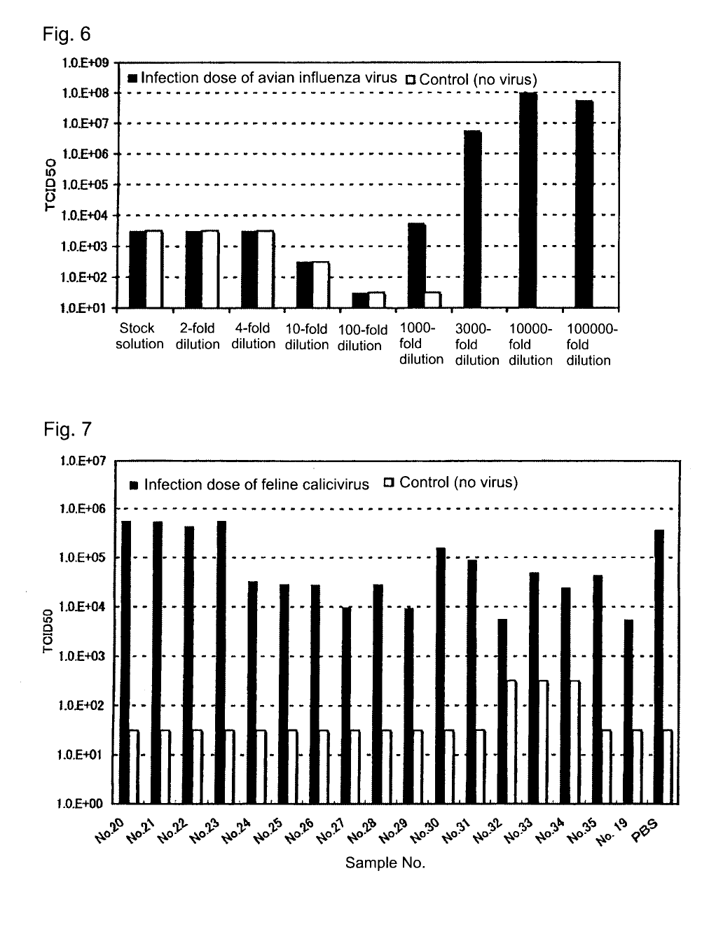

The results of examining the effect of the concentration of the antiviral agent (sample No. 19) on the ability to inactivate the feline calicivirus (Example 6) are shown in FIG. 5. Likewise, the results of examining the effect of the concentration of the antiviral agent (sample No. 19) on the ability to inactivate the avian influenza virus (Example 6) are shown in FIG. 6.

The antiviral agent diluted to 100 times (soap concentration: 0.0035 M) reduced the infection dose to one-hundredth, and the antiviral agent diluted to 10 times (soap concentration: 0.035 M) reduced the infection dose to one-thousandth in the infectivity of the feline calicivirus.

The antiviral agent diluted to 1000 times (soap concentration: 0.00035 M) reduced the infection dose to one-thousandth in the infectivity of the avian influenza virus. The cause is thought to be a difference in effects that the feline calicivirus has no envelop different from the avian influenza virus.

The soap at a concentration of 2.5% by weight or less has no cleansing function. Thus, this effect is thought to be derived from something different from the cleansing function of the soap.

### Example 7

### Comparison in Various Additives

Additives often used for the cleansing agent were examined.

First, the ability to inactivate the virus was compared between the case of the antiviral agent (sample No. 19) alone prepared in Example 6 and the case of adding the additive thereto. The additives used and a combination rate thereof are shown in Table 3.

TABLE 3

| Sample | | Combination |
|---|---|---|
| No. | Additive | rate (%) |
| 20 | Disodium ethylenediamine tetraacetate | 0.5 |
| 21 | Tetrasodium ethylenediamine tetraacetate | 0.5 |
| 22 | Methyl parahydroxybenzoate | 0.5 |
| 23 | Propyl parahydroxybenzoate | 0.5 |
| 24 | Butyl parahydroxybenzoate | 0.5 |
| 25 | 2,4,4′-Trichloro-2′-hydroxydiphenyl | 0.3 |
| | ether | |
| 26 | Isopropylmethylphenol | 0.3 |
| 27 | Phenoxyethanol | 0.5 |
| 28 | 1,2-Hexanediol | 0.5 |
| 29 | 1,2-Propylene glycol | 10 |
| 30 | 1,3-Butylene glycol | 10 |
| 31 | Ethanol | 10 |
| 32 | Glycerin | 10 |
| 33 | O-[2-Hydroxy-3-(trimethylammonio)propyl] | 1 |
| | hydroxyethylcellulose chloride | |
| 34 | Coconut oil fatty acid monoethanolamide | 5 |
| 35 | Lauryldimethylaminoacetic acid betaine | 5 |

TABLE 4

| Sample | | Combination |
|---|---|---|
| No. | Surface-active agent | rate (%) |
| 36 | Ammonium lauryl sulfate | 10.32 |
| 37 | Sodium polyoxyethylene alkyl ether sulfate | 10.32 |

The antiviral agent (sample No. 19) (total soap concentration: 0.35 M) was combined with the additive described in Table 3 at a weight ratio described in Table 3, and the ability of the combination to inactivate the virus was examined. For comparison, the case of not adding the additive was also examined.

The ability to inactivate the feline calicivirus was measured in the same manner as in Example 1, and the ability to inactivate the avian influenza virus was measured in the same manner as in Example 2. For comparison, the case of not adding the additive was also examined.

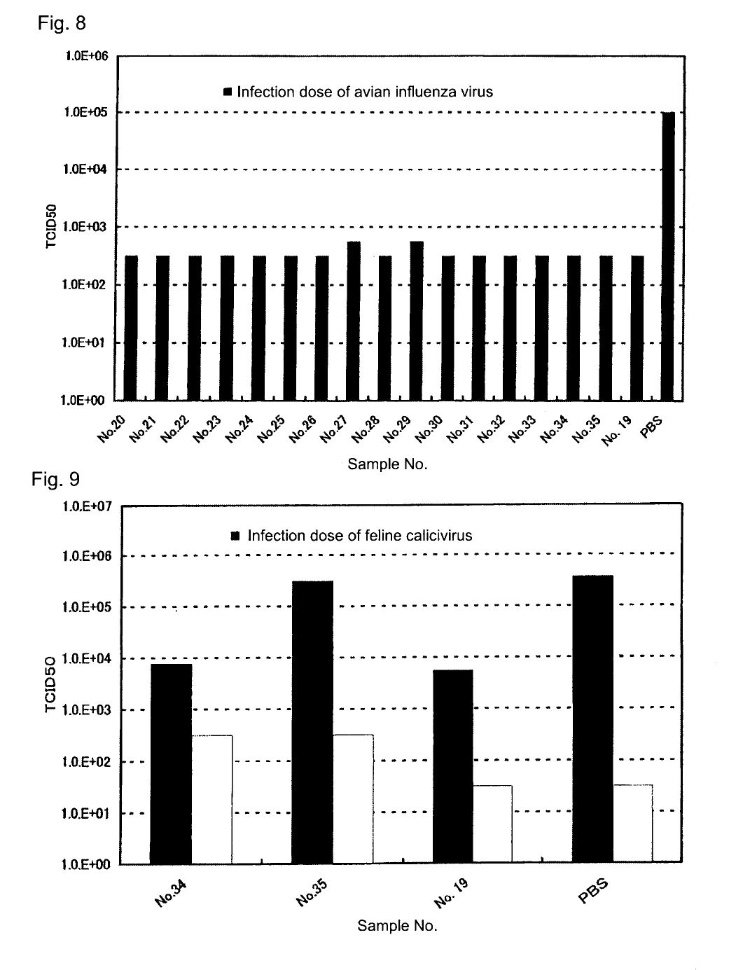

The results of measuring the ability to inactivate the feline calicivirus in the case of the antiviral agent (sample No. 19) and the cases of adding the various additives thereto are shown in FIG. 7. Likewise, the results of measuring the ability to inactivate the avian influenza virus in the case of the antiviral agent (sample No. 19) and the cases of adding the various additives thereto are shown in FIG. 8. The solid bars denote the remaining infection dose of virus, and the open bars denote the cytotoxicity. A horizontal axis denotes the sample number. The bars on the extreme right show the case of using PBS, and indicate the viral infection dose when no antiviral agent was added, i.e., the infection dose of the virus used in the experiment.

There was no additive compared that enhanced the ability of the antiviral agent (sample No. 19) of the present invention to inactivate the virus. There were some additives such as coconut oil fatty acid monoethanolamide, which enhanced the cytotoxicity. It was shown that attention is required for use of the additive.

### Example 8

### Comparison with Surface-Active Agent Other than Soap

The surface-active agents other than the soap, which were often combined in the cleansing agent, were examined.

The ability to inactivate the virus was compared between the antiviral agent (sample No. 19) alone prepared in Example 6 and the surface-active agents other than the soap. The surface-active agents used and the combination rate thereof are shown in Table 4.

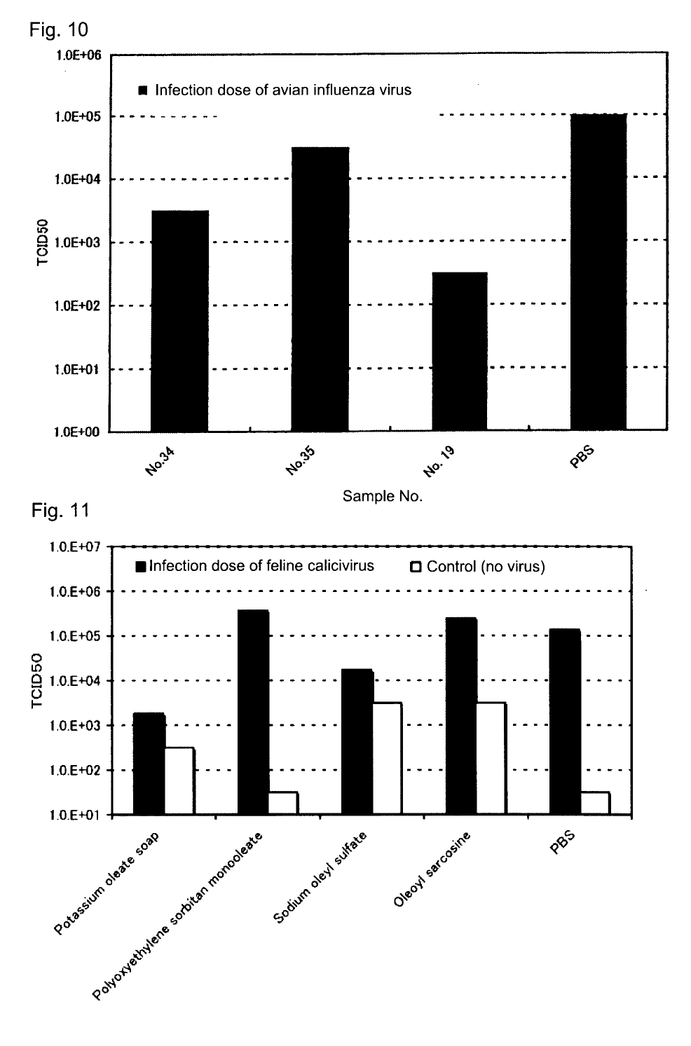

The results of measuring the ability of the antiviral agent (sample No. 19) and the surface-active agents other than the soaps to inactivate the feline calicivirus are shown in FIG. 9. Likewise, the results of measuring the ability of the antiviral agent (sample No. 19) and the surface-active agents other than the soaps to inactivate the avian influenza virus are shown in FIG. 10. The solid bars denote the remaining infection dose of virus, and the open bars denote the cytotoxicity. The horizontal axis denotes the sample number. The bars on the extreme right show the case of using PBS, and indicate the viral infection dose when no antiviral agent was added, i.e., the infection dose of the virus used in the experiment.

There was no surface-active agent compared that exhibited the virus inactivation ability equivalent to that of the antiviral agent (sample No. 19) of the present invention, and the fatty acid soap of the present invention exhibited the most excellent virus inactivation ability. It was shown that sodium polyoxyethylene alkyl ether sulfate had low antiviral activity in addition to high cytotoxicity and thus attention was required for use thereof.

### Example 9

### Comparison with Various Surface-Active Agents Having C18 Unsaturated Alkyl Chain

The ability to inactivate the virus was compared between various surface-active agents having the C18 unsaturated alkyl chain and the potassium oleate soap (C18:1) prepared in Example 1. Polyoxyethylene sorbitan monooleate (nonionic surface-active agent), sodium oleyl sulfate (anionic surface-active agent), and oleoyl sarcosine (anionic surface-active agent) were compared with the potassium oleate soap. They were diluted to 0.0035 mol/L with purified water for use.

The ability to inactivate the feline calicivirus was examined in the same manner as in Example 1, except for using the potassium oleate soap (C18:1) prepared in Example 1 or any one of the above three synthetic surface-active agents as the antiviral agent. Further, the ability to inactivate the avian influenza virus was examined in the same manner as in Example 2.

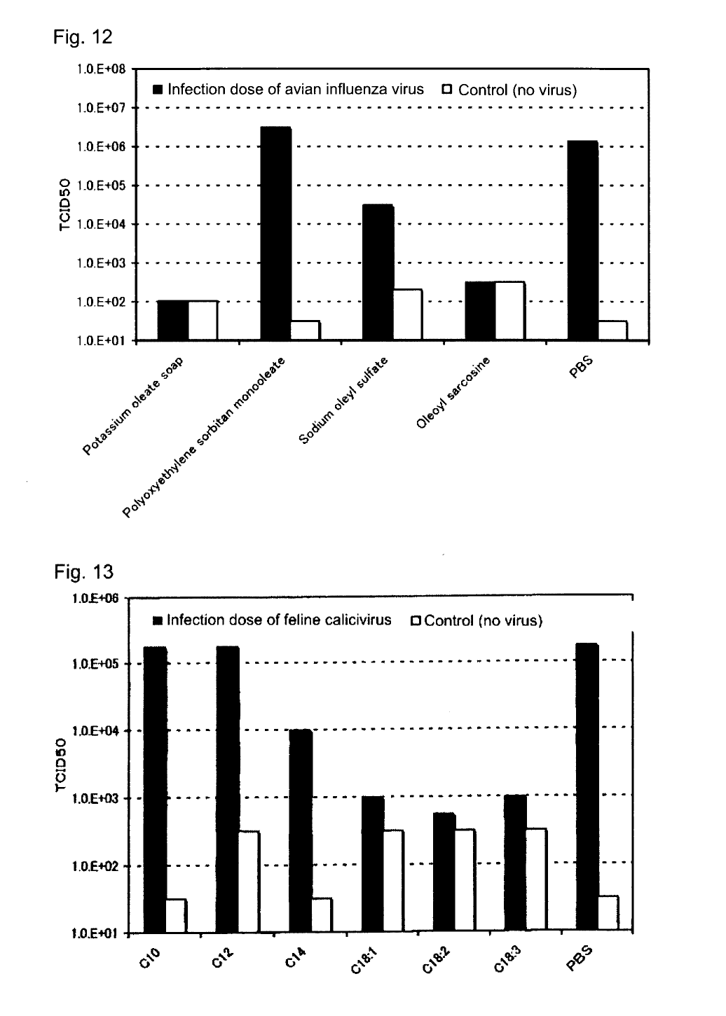

The results in Example 9 are shown in FIG. 11 and FIG. 12. The effects on the feline calicivirus and the effects on the avian influenza virus are shown in FIG. 11 and FIG. 12, respectively.

Polyoxyethylene sorbitan monooleate that was the nonionic surface-active agent in the synthetic surface-active agents having the C18 unsaturated alkyl group was shown to have no ability to inactivate either of the viruses. In the anionic surface-active agent, the infection dose of the avian influenza virus was reduced to one-tenth or less by sodium oleyl sulfate and to about one-thousandth by oleoyl sarcosine, but the infection dose of the feline calicivirus was reduced to one-tenth or less by sodium oleyl sulfate, but could not be reduced by oleoyl sarcosine. Therefore, it was shown that the anionic surface-active agent exhibited the antiviral activity in the surface-active agent having the C18 unsaturated alkyl group and that the potassium oleate soap (C18:1) that was the fatty acid soap had excellent antiviral activity among them.

### Example 10

### Difference in Antiviral Activity Due to Carbon Number and Unsaturation Degree in Fatty Acid Chain

Potassium soaps of the linoleic acid (C18:2) and the linolenic acid (C18:3) were prepared in the same manner as in Example 1, in addition to the potassium soaps of the lauric acid (C12), the myristic acid (C14) and the oleic acid (C18:1) prepared in Example 1. The ability to inactivate the feline calicivirus was examined in the same manner as in Example 1, except that each soap was diluted to the concentration of 0.035 M to use as the antiviral agent. Further, the ability to inactivate the avian influenza virus was examined in the same manner as in Example 2.

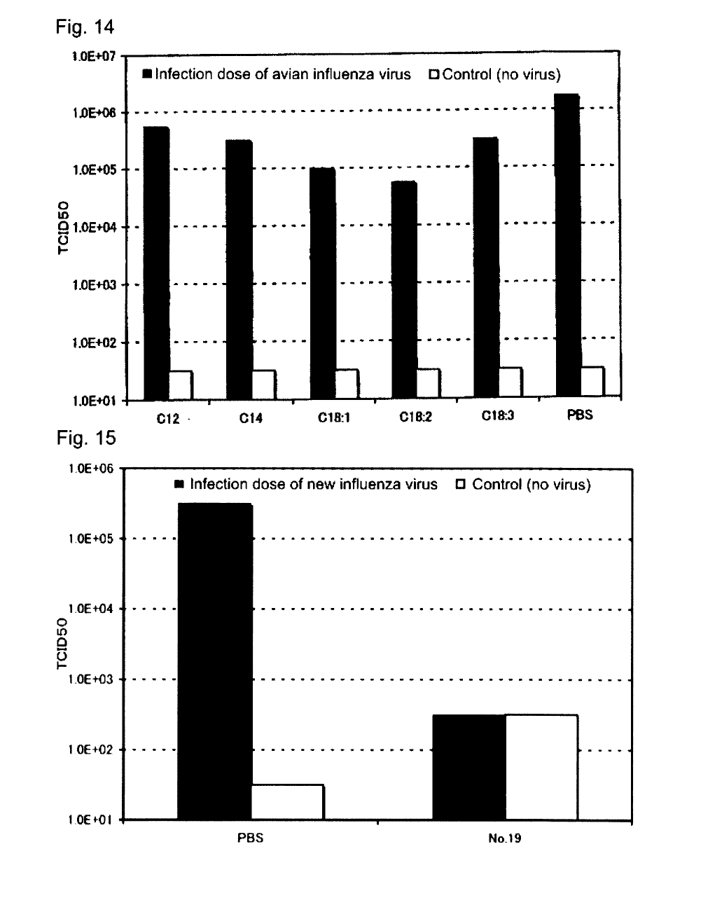

The results in Example 10 are shown in FIG. 13 and FIG. 14. The effects on the feline calicivirus and the effects on the avian influenza virus are shown in FIG. 13 and FIG. 14, respectively.

It was shown that the ability to inactivate either of the viruses was increased as the carbon number was increased in the fatty acid chain in the fatty acid soap. It was also shown that the C18 unsaturated fatty acid soaps had excellent ability to inactivate the feline calicivirus. For the avian influenza virus, it was shown that the effect of the C18:3 (linolenate) soap was weak and the effect of the C18:1 (oleate) or the C18:2 (linoleate) soap was excellent.

### Example 11

### Effect on Swine-Origin Influenza a (H1N1)

Using the sample No. 19 prepared in Example 7, the antiviral effect on a new influenza virus (swine-origin influenza A/Hiroshima/201/2009 (H1N1)) that had caused a pandemic in 2009 was examined.

The antiviral effect was measured in the same manner as in Example 2, except that the sample No. 19 prepared as the antiviral agent in Example 6 was diluted to 100 times to make a 0.0035 M solution and the swine-origin influenza A/Hiroshima/201/2009 (H1N1) strain (supplied by Hiroshima Prefectural Technology Research Institute) was used as the virus.

The results in Example 11 are shown in FIG. 15. It was shown that the antiviral agent of the sample No. 19 prepared in Example 6 had an excellent effect on the new influenza virus.

### INDUSTRIAL APPLICABILITY

The present invention may provide the antiviral agent that cleanses the hands and face, etc., does not require wiping off or rinsing off with water, inactivates viruses such as a norovirus and influenza virus, and is excellent in germicidal properties. The present invention may also provide the antiviral agent that may be impregnated in cloth, etc., for use as a wiper and a mask, added in a foot washing bath or impregnated in a foot wipe mat for use, inactivates viruses such as a norovirus and influenza virus, and is excellent in germicidal properties. Further, the present invention may provide the cleansing agent that may be used safely for the hands and feet and face as the cleansing agent, and inactivates viruses such as a norovirus and influenza virus, and is excellent in germicidal properties.

1. An antiviral agent, comprising:
a laurate soap;
a myristate soap; and
an oleate soap,
wherein a percentage of a summed amount of the laurate soap and the myristate soap is 70% by weight or more based on a total amount of saturated fatty acid soaps,
wherein a percentage of a stearate soap and a percentage of a palmitate soap are less than 15% by weight based on the total amount of the saturated fatty acid soaps, and
wherein a percentage of the oleate soap is 50 to 75% by weight based on a total amount of soaps.

2. The agent of to claim 1, wherein a percentage of the soaps in the antiviral agent is 0.5 to 40% by weight.

3. The agent of claim 1, mainly comprising soaps comprising a fatty acid at least one counterion selected from the group consisting of potassium, sodium, arginine salt, ammonium, and triethanolammonium.

4. The agent of claim 1, wherein an inactivation ability against viral infectivity of an influenza virus reduces a viral infectivity titer to 1% or less.

5. The agent of claim 1, wherein an inactivation ability against viral infectivity of a feline calicivirus reduces a viral infectivity titer to 1% or less.

6. A cleansing agent, comprising the antiviral agent of claim 1.

7. The agent of claim 2, mainly comprising soaps comprising a fatty acid at least one counterion selected from the group consisting of potassium, sodium, arginine salt, ammonium, and triethanolammonium.

8. The agent of claim 2, wherein an inactivation ability against viral infectivity of an influenza virus reduces a viral infectivity titer to 1% or less.

9. The agent of claim 3, wherein an inactivation ability against viral infectivity of an influenza virus reduces a viral infectivity titer to 1% or less.

10. The agent of claim 7, wherein an inactivation ability against viral infectivity of an influenza virus reduces a viral infectivity titer to 1% or less.

11. The agent of claim 2, wherein an inactivation ability against viral infectivity of a feline calicivirus reduces a viral infectivity titer to 1% or less.

12. The agent of claim 3, wherein an inactivation ability against viral infectivity of a feline calicivirus reduces a viral infectivity titer to 1% or less.

13. The agent of claim 7, wherein an inactivation ability against viral infectivity of a feline calicivirus reduces a viral infectivity titer to 1% or less.

14. A cleansing agent, comprising the antiviral agent of claim 2.

15. A cleansing agent, comprising the antiviral agent of claim 3.

16. A cleansing agent, comprising the antiviral agent of claim 4.

17. A cleansing agent, comprising the antiviral agent of claim 5.

18. A cleansing agent, comprising the antiviral agent of claim 7.

19. A cleansing agent, comprising the antiviral agent of claim 8.

20. A cleansing agent, comprising the antiviral agent of claim 9.
